(19) United States
(12) Patent Application Publication
(10) Pub. No.: US 2022/0175268 A1
(43) Pub. Date: Jun. 9, 2022

(54) LESION CHARACTERIZATION PROCESSES

(71) Applicant: Medtronic, Inc., Minneapolis, MN (US)

(72) Inventors: Megan M. Schmidt, Blaine, MN (US); Lars M. Mattison, St. Anthony, MN (US); Anthony Rorvick, Champlin, MN (US); Christina M. Crassas, Minnetonka, MN (US)

(21) Appl. No.: 17/540,548

(22) Filed: Dec. 2, 2021

Related U.S. Application Data
(60) Provisional application No. 63/120,965, filed on Dec. 3, 2020.

(51) Int. Cl. A61B 5/06; A61B 5/00; A61B 5/318

(52) U.S. Cl. CPC A61B 5/063; A61B 5/7264; A61B 5/726; A61B 5/7257; A61B 5/318

(57) ABSTRACT

A lesion characterization process is disclosed. According to one aspect, a method includes obtaining measurements of at least one of an impedance magnitude, impedance phase, a temperature, and electrical properties of tissue of the lesion. The method further includes determining at least one lesion property including at least one of a depth of the lesion, percent transmurality of the lesion, lesion surface area and lesion volume based on at least one of the obtained measurements.

### CROSS-REFERENCE TO RELATED APPLICATION

This application claims the benefit of U.S. Application Ser. No. 63/120,965, filed Dec. 3, 2020.

### FIELD

The present technology is generally related to lesion characterization processes.

### BACKGROUND

Cardiac ablation is a procedure that may be employed to treat an irregular heart rhythm. Cardiac ablation works by scarring heart tissue to disrupt faulty electrical signals causing the arrhythmia. In recent years many radio frequency (RF) ablation systems have relied heavily upon the use of contact force (CF) sensing to ensure quality lesion formation. Determining the size of a lesion formed by an RF ablation system is desirable, yet difficult. With CF sensing, algorithms have been developed that incorporate a force-time integral to determine lesion effectiveness. Further, algorithms have been developed that include weighting of CF, power and duration.

### SUMMARY

The techniques of this disclosure generally relate to lesion characterization processes. Algorithms are presented that utilize measurements of impedance magnitude and phase, temperature and electrical properties of the tissue to characterize a lesion. A time-temperature integral may also be calculated. The electrical properties of the tissue may be included in or derived from electrocardiogram (EGM) waveforms and monophasic action potential (MAP) waveforms. In some embodiments, measurements are made before ablation, during ablation and after ablation. Combinations of these measurements are input to a specially programmed computer to evaluate lesion characteristics including lesion depth, percent transmurality, surface area and volume. This information about the lesion may be fed back to a practitioner who may make an assessment of the effectiveness of the lesion.

According to one aspect, a method of characterizing a lesion based on a plurality of measurements is provided. The method includes obtaining measurements of at least one of an impedance magnitude, impedance phase, a temperature, and electrical properties of tissue of the lesion. The method also includes performing a statistical analysis of the obtained measurements. The method also includes determining at least one lesion property including at least one of a depth of the lesion, percent transmurality of the lesion, lesion surface area and lesion volume based at least in part on the statistical analysis of the obtained measurements.

According to this aspect, in some embodiments, the electrical properties of the tissue of the lesion include at least one of electrocardiogram (EGM) waveform and monophasic action potential (MAP) waveform properties. In some embodiments, determining at least one lesion property includes calculating the depth of the lesion from an equation that includes as inputs at least one of: an impedance phase change over a range of impedance phase measurements; a peak to peak impedance magnitude change over a range of impedance magnitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion surface area from an equation that includes as inputs at least one of: a percent change in phase of a measured impedance; an average peak to peak value of post-ablation measured impedance; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion volume from an equation that includes as inputs at least one of: an average of post ablation impedance magnitude measurements; an average of post ablation impedance phase measurements; an average of changes in peak to peak impedance amplitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating a transmurality of the lesion from an equation that includes as inputs at least one of: a percent change in impedance magnitude; a percent change in impedance phase; a maximum temperature measurement; changes in electrical properties of tissue of the lesion; and a sum of temperature measurements. In some embodiments, the equation is determined based at least in part on a statistical analysis of a plurality of the measurements of the at least one of an impedance magnitude, impedance phase, a temperature, and changes in electrical properties of tissue of the lesion. In some embodiments, determining at least one lesion property includes applying one of a Fourier transform, a filter and a wavelet matching algorithm to at least one of the obtained measurements. In some embodiments, determining at least one lesion property is further based on at least one of a tissue thickness, a distance of a probe to the tissue of the lesion. In some embodiments, at least one of the tissue thickness and the distance is determined by at least one of an electro-anatomical mapping system, medical imaging and a dielectric method.

According to another aspect, a method of characterizing a lesion based on a plurality of measurements is provided. The includes obtaining measurements of at least one of an impedance magnitude, impedance phase, a temperature, and electrical properties of tissue of the lesion. The method also includes performing a statistical analysis of the obtained measurements. The method also includes determining at least one lesion property including at least one of a depth of the lesion, percent transmurality of the lesion, lesion surface area, lesion volume and lesion contiguity based at least in part on the statistical analysis of the obtained measurements.

According to this aspect, in some embodiments, the electrical properties of the tissue of the lesion include at least one of electrocardiogram (EGM) waveform and monophasic action potential (MAP) waveform properties. In some embodiments, determining at least one lesion property includes calculating the depth of the lesion from an equation that includes as inputs at least one of: an impedance phase change over a range of impedance phase measurements; a peak to peak impedance magnitude change over a range of impedance magnitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion surface area from an equation that includes as inputs at least one of: a percent change in phase of a measured impedance; an average peak to peak value of post-ablation measured impedance; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion volume from an equation that includes as inputs at least one of: an average of post ablation impedance magnitude measurements; an average of post ablation impedance phase measurements; an average of changes in peak to peak impedance amplitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating a transmurality of the lesion from an equation that includes as inputs at least one of: a percent change in impedance magnitude; a percent change in impedance phase; a maximum temperature measurement; changes in electrical properties of tissue of the lesion; and a sum of temperature measurements. In some embodiments, the equation is determined based at least in part on a statistical analysis of a plurality of the measurements of the at least one of an impedance magnitude, impedance phase, a temperature, and changes in electrical properties of tissue of the lesion. In some embodiments, determining at least one lesion property includes applying one of a Fourier transform, a filter and a wavelet matching algorithm to at least one of the obtained measurements. In some embodiments, determining at least one lesion property is further based on at least one of a tissue thickness, a distance of a probe to the tissue of the lesion. In some embodiments, at least one of the tissue thickness and the distance is determined by at least one of an electro-anatomical mapping system, medical imaging and a dielectric method.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a block diagram of an ablation system constructed according to principles set forth herein;

FIG. 2 is a drawing of an ablation catheter of the ablation system of FIG. 1;

FIG. 3 is an illustration of a flow of inputs to an algorithm for lesion characterization;

FIG. 4 is an illustration of inputs to a data acquisition system before, during and after ablation;

FIG. 5 is a block diagram of a data acquisition system;

FIG. 6 is one example of action potential durations;

FIG. 7 is a flowchart of an example process for development of models to characterize a lesion; and

FIG. 8 is a flowchart of an example process for characterizing a lesion.

### DETAILED DESCRIPTION

The present disclosure relates to lesion characterization processes. More particularly, the present disclosure relates to characterization of lesions created by RF ablation or pulsed electric field (PEF) ablation systems creating lesions via irreversible electroporation. The lesion characterization system characterizes lesion depth, surface area, volume and/or transmurality, without the use of contact force. Transmurality may be defined as an extent of penetration into the wall of the heart. For example, a fully transmural lesion spans the entire thickness of the myocardial tissue.

The techniques of this disclosure may be used to generate lesion characterizations without the use of surrogates that are not directly associated with lesion formation. For example, according to aspects of this disclosure, lesions can be characterized without evaluations of contact force sensing or use of a time-force integral. The techniques may provide more accurate representations of lesion characteristics, such as lesion depth and/or lesion transmurality. In some instances, lesion characterizations may be generated and output throughout an ablation procedure, e.g., a plurality of times to provide a real-time indication of lesion formation during the ablation procedure. In this way, the techniques of this disclosure may be used to provide an accurate representation of lesion formation during an ablation procedure, which may provide a reference to determine when to stop ablating tissue (e.g., when a lesion is sufficiently formed to prevent unwanted electrical impulses).

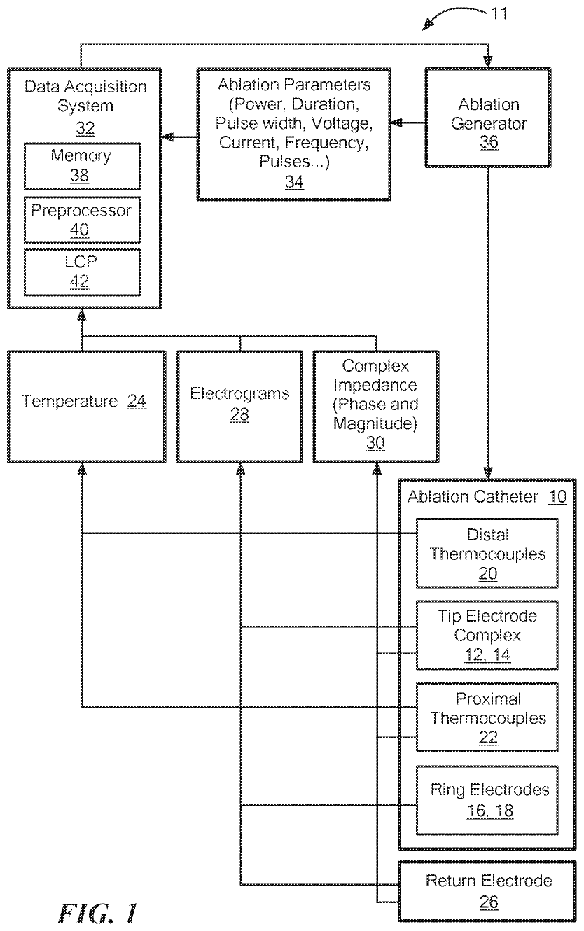

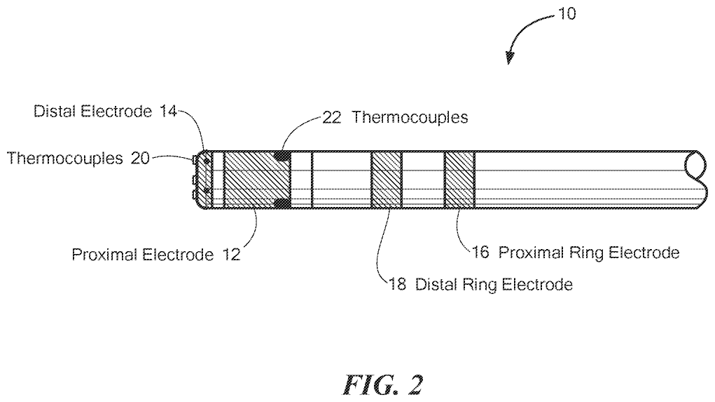

FIG. 1 illustrates an ablation catheter 10 in an ablation system 11 constructed according to principles set forth herein. The ablation catheter 10 is configured to perform electrical measurements and temperature measurements of tissue that is to be ablated or has been ablated. The electrical measurements are made by the electrodes 12, 14, 16 and 18 and the temperature measurements are made by thermocouples 20 and 22, as described below with reference to FIG. 2. The electrodes 12, 14, 16 and 18 and/or other electrodes on the ablation catheter 10 may be configured to apply RF or PEF ablation therapy.

FIG. 1 also shows a data acquisition system 32 that is configured to acquire or receive the electrical, temperature measurements as well as electrograms 28 and complex impedance 30. These measured parameters may be processed to produce calculated parameters. The calculated parameters are statistically analyzed to determine lesion characteristics such as area, depth, volume and transmurality.

The data acquisition system 32 also receives ablation parameters 34 that may include one or more of power, duration, pulse width, voltage, frequency, number of pulses, etc., and/or other parameters from an ablation generator 36. These parameters may be input by the operator of the ablation system 11. The ablation generator 36 may be a commercially available ablation generator or may be specially constructed to perform ablation generator functions as described herein. For example, the ablation generator 36 may be configured to provide pulses of RF energy, delivered to the cardiac tissue to cause ablation. The delivery of the RF energy may be by direct electrical contact and/or by electromagnetic energy. For example, some embodiments, the ablation generator 36 may provide laser ablation. In some embodiments, the ablation generator 36 may provide cryoablation. In some embodiments, the ablation generator 36 may provide PEF ablation or RF ablation. Any one or more ablation energy sources may be employed, of the same type or of different types. The ablation parameters 34 may be input by an input device (such as a keyboard, mouse, etc.) of the data acquisition system 32, the data acquisition system 32 transmits such inputs to the ablation generator 36. In some embodiments the ablation parameters are input to the ablation generator 36, and these inputs are transmitted from the ablation generator to the data acquisition system 32. The ablation generator 36 may output pulsed electric fields or RF energy, for example, to the catheter 10 to be applied to the cardiac tissue to form the lesion.

The data acquisition system 32 includes a memory 38, a preprocessor 40 and lesion characteristics predictor (LCP) 42. LCP 42 may be described as a “predictor,” because, while the measurements described herein do not provide a direct measurement, for example, lesion characteristics such as lesion depth or transmurality, LCP 42 may be used to determine an accurate representation (or “prediction”) of such characteristics. The memory 38 is configured to store parameters calculated by the preprocessor 40 and to store lesion characteristics generated by the LCP 42. The preprocessor 40 calculates parameters based on measured data that are used by the LCP 42 to determine lesion characteristics.

According to aspects of this disclosure, as described in greater detail below, data acquisition system 32 may be configured to generate lesion characterizations without the use of certain surrogates, such as force measurements. Rather, LCP 42 may rely on ablation parameters 32, which may allow data acquisition system 32 to accurately output lesion depth and/or lesion transmurality. In some instances, data acquisition system 32 may generate and output lesion characteristics, e.g., via LCP 42, throughout an ablation procedure, e.g., a plurality of times to provide a real-time indication of lesion formation during the ablation procedure.

While described with respect to separate components in FIG. 1 for purposes of illustration, it should be understood that, in some examples, data acquisition system 32 (or a sub-set of components of data acquisition system 32) may be incorporated in ablation generator 36.

FIG. 2 illustrates one example ablation catheter 10 that can be used for PEF and/or RF ablation and measurement. The catheter 10 includes a proximal electrode 12, a distal electrode 14, a proximal ring electrode 16, a distal ring electrode 18, distal thermocouples 20 and proximal thermocouples 22. Tissue electrical activity may be recorded as measured between the distal electrode 14 and second electrode 12, 16 or 18, for example, or any combination thereof. Tissue electrical activity may also be recorded between any of the catheter electrodes (12, 14, 16, and 18) and a common reference such as Wilson's Central Terminal. Catheter distal and proximal thermocouples 20 and 22 sense temperature. In this example, both thermocouples 20 and 22 may be two of 3 thermocouples.

Power, voltage and/or current may be measured between the proximal and distal electrodes 12 and 14, for example. From measurements of voltage and current, a magnitude and phase angle of impedance can be calculated. Signals from the distal and proximal thermocouples 20 and 22 provide temperature 24. Signals from the tip electrodes (proximal electrode 12 and distal electrode 14) and a voltage reference level from a return electrode 26 provide electrograms 28. The electrograms 28 may include bipolar and/or unipolar electrograms as well as monophasic action potentials. Signals from the tip electrodes 12 and 14 a voltage reference level from the return electrode 26 provide complex impedance 30 (magnitude and phase angle) values. Complex impedance values may be collected at a plurality of frequencies to provide a frequency response that may be compared to a frequency domain representation of the output of the ablation generator 36.

According to aspects of this disclosure, the configuration of catheter 10 may allow for focal measurements of impendence and ablation parameters. For example, the spacing between the electrodes 12 and 14 is relatively small, and in combination with the small size of electrode 14, the readings are much more focal than a differently configured catheter. The same is true when evaluating the electrogram (EGM) and monophasic action potential (MAP) recordings.

Catheter 10 also enables higher accuracy of temperature recording at the tissue-device interface. For example, catheter 10 may be configured similarly to the DiamondTemp™ ablation system manufactured by Medtronic Inc. and may include one or more diamond elements included in the interior of catheter 10 at the distal end. At least one such configuration is described in U.S. Publication No. 2018/0289284, assigned to Epix Therapeutics, and incorporated by reference in its entirety. At a high level, diamonds in the tip of catheter 10 may allow heat to be shunted away from tissue being ablated and may provide for more uniform heating of the tip. This uniform heating and heat shunting allows for catheter 10 to operate at a lower irrigation rate (less saline being injected through the catheter during ablation). Lower irrigation allows for more accurate temperature recordings from thermocouples 22.

According to aspects of this disclosure, catheter 10 may output temperature sensed via thermocouples 20 and 22 and signals from one or more electrodes 12, 14, 16, and/or 18 to generate at least one of an impedance magnitude, impedance phase, and electrical properties (e.g., EGM and/or MAP recordings) of tissue to generate lesion characterizations without the use of certain surrogates, such as force measurements. In some examples, the characterizations may be formed throughout an ablation procedure, e.g., while catheter 10 is applying energy to ablate tissue.

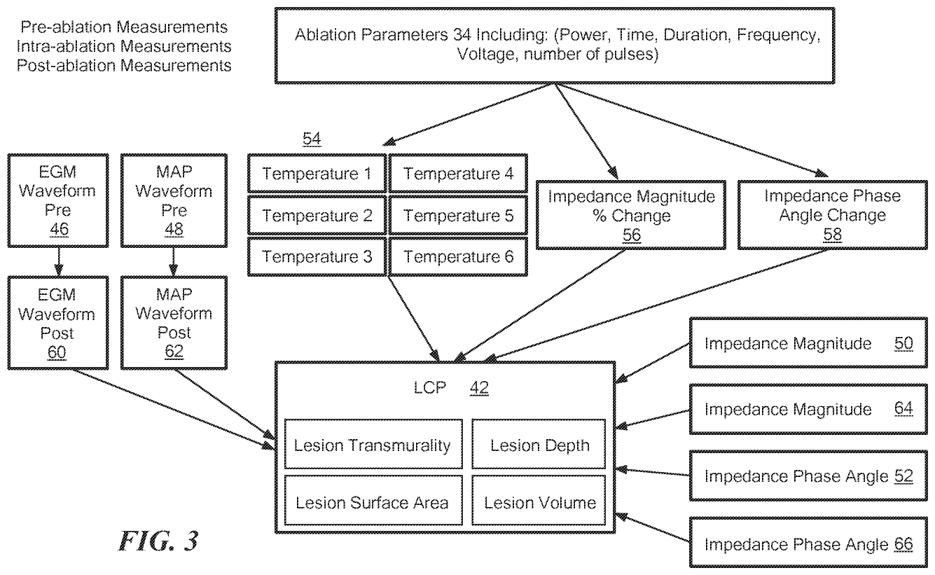

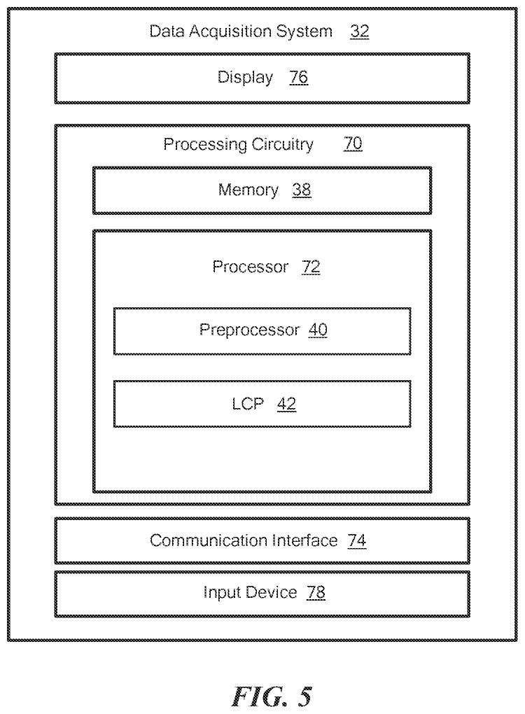

FIG. 3 is a flowchart of the data gathering by a lesion characterization algorithm 37 which may be implemented in the data acquisition system 32, for example, by a processor executing software instructions to process measurement data and ablation generation parameters, as described below with respect to FIG. 5. The lesion characterization prediction algorithm 42 may compute lesion parameters that include one or more of lesion volume, surface area, depth and transmurality. The lesion parameters are based on measurements that are taken before ablation (pre-ablation), measurements taken during ablation (intra-ablation) and measurements taken after ablation (post ablation).

The pre-ablation measurements may include electrocardiogram (EGM) waveforms 46, MAP waveforms 48, impedance magnitude 50 and impedance phase angle 52. The intra-ablation measurements may include temperatures 54, impedance percent change 56 and impedance phase angle change 58. Intra-ablation measurements may be based on the ablation parameters 34. The post-ablation measurements may include EGM waveforms 60, MAP waveforms 62, impedance magnitude 64 and impedance phase angle 66. Using these pre-ablation, intra-ablation, and post-ablation measurements, lesion depth and transmurality can be determined without force sensing or reliance on impedance drop correlations to determine depth.

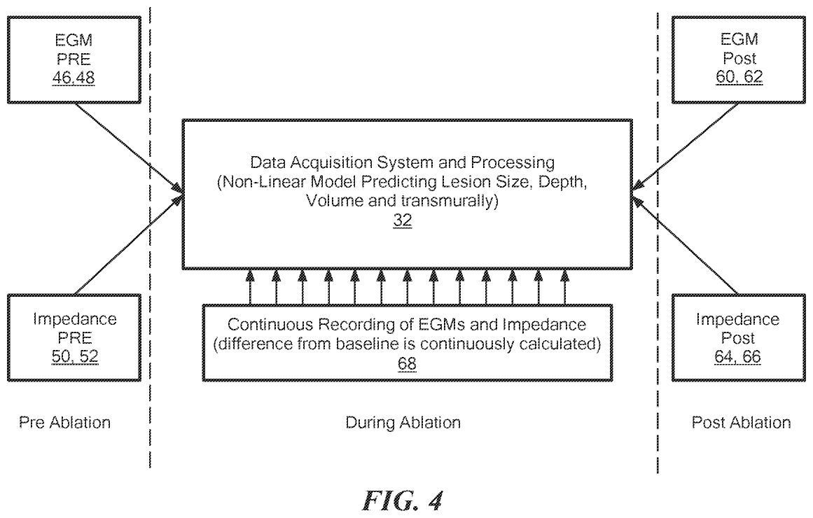

FIG. 4 is an illustration of the data acquisition system 32 receiving inputs from pre-ablation parameters 46, 48, 50 and 52, receiving inputs from continuous EGM and impedance parameters 66 during ablation, and receiving inputs from post-ablation parameters 60, 62, 64 and 66. Any one or more of the inputs received by the data acquisition system 32 may be used to develop a non-linear model to determine lesion size (or surface area), depth, volume and transmurality. In other words, the surface area, depth, volume and transmurality of a lesion may be determined based on a statistical analysis of data from a plurality of previously measured lesions. The LCP unit 42 performs a process for calculating the lesion characteristics based on a previously performed statistical analysis of previously measured lesions. The statistical analysis may be performed using commercially available software such as Design Expert by StatEase®. The statistical analysis algorithm outputs values called residuals, which are evaluated to determine whether another curve or equation provides a better fit to the remaining inputs. This is done for each lesion characteristic (e.g., surface area, volume, depth, transmurality).

According to aspects of this disclosure, LCP unit 42 may perform the calculations described herein prior to and during an ablation procedure. For example, LCP unit 42 may continue to generate and output lesion characteristics, which may be referenced to determine when a lesion has formed to a sufficient degree (e.g., such that the lesion blocks electrical impulses).

FIG. 5 is a block diagram of an example embodiment of the data acquisition system 32. The data acquisition system 32 includes processing circuitry 70, which may include the memory 38 and a processor 72. Processing circuitry 70 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 72 may be configured to access (e.g., write to and/or read from) the memory 38, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

The processing circuitry 70 may be configured to control any of the methods and/or processes described herein and/or to cause such methods, and/or processes to be performed. Processor 72 may comprise multiple processors. The memory 38 is configured to store data, programmatic software code and/or other information described herein. In some embodiments, the software stored in the memory 38 may include instructions that, when executed by the processor 72, respectively, and/or processing circuitry 70, respectively, causes the processor 72, and/or processing circuitry 70 to perform the processes described herein with respect to the data acquisition system 32.

In addition to or instead of a processor 72, such as a central processing unit, and memory, the processing circuitry 70 may comprise integrated circuitry for processing and/or control, e.g., one or more processors and/or processor cores and/or FPGAs (Field Programmable Gate Array) and/or ASICs (Application Specific Integrated Circuitry) adapted to execute instructions. The processor 72 may be configured to access (e.g., write to and/or read from) the memory 38, which may comprise any kind of volatile and/or nonvolatile memory, e.g., cache and/or buffer memory and/or RAM (Random Access Memory) and/or ROM (Read-Only Memory) and/or optical memory and/or EPROM (Erasable Programmable Read-Only Memory).

The processing circuitry 70 may be configured to control any of the methods and/or processes described herein and/or to cause such methods, and/or processes to be performed, e.g., by the data acquisition system 32. The memory 38 is configured to store data, programmatic software code and/or other information described herein. In some embodiments, the software executed by the processor 72 may include instructions that, when executed by the processor 72 and/or processing circuitry 70, causes the processor 72 and/or processing circuitry 70 to perform the processes described herein with respect to the data acquisition system 32.

The data acquisition system 32 may also include a communication interface 74 configured to receive the measured parameters from the electrodes 12, 14, 16 and 18 and thermocouples 20, 22. The communication interface 74 may also be configured to send ablation parameters to the ablation generator 36. The data acquisition system 32 may also include a display 76 (and/or an audio speaker) to display information and an input device 78, such as a keyboard and/or mouse to receive input from an operator or user of the data acquisition system 32.

The memory 38 may include software modules executable by the processor 72. These software modules may instruct the processor 72 to perform functions attributable to one or more of the following modules: preprocessor 40, and lesion characteristics predictor (LCP) 42. The preprocessor 40 receives the measured parameters from the electrodes 12, 14, 16 and 18 and thermocouples 20, 22 and uses one or more of these parameters to compute one or more of the following calculated parameters: an average post ablation impedance magnitude, a difference between a post-ablation impedance magnitude and a pre-ablation impedance magnitude. The LCP 42 may determine lesion parameters such as depth and transmurality based on these calculated parameters. The measured parameters may also be processed by the preprocessor 40 to compute one or more of the following calculated parameters: an average peak to peak impedance magnitude, a change in peak to peak impedance magnitude and a percent change in peak to peak impedance magnitude.

Similarly, the measured parameters may also be processed by the preprocessor 40 to compute one or more of the following calculated parameters: an average peak to peak impedance phase angle, a change in peak to peak impedance phase angle and a percent change in peak to peak impedance phase angle. Impedance phase angle may be measured at the same or a different frequency (such as 826 kHz) than the frequency at which the impedance magnitude is measured.

A “low” frequency such as 46 kHz will allow measurements that are more sensitive to changes in the extracellular matrix. A “high” frequency such as 826 kHz may be more sensitive to changes in the intracellular matrix. Recording impedances at different excitation wavelengths can have varying levels of sensitivity to scarred (ablated) myocardium tissue versus healthy tissue.

The temperatures measured by the thermocouples may be processed by the preprocessor 40 to compute a maximum temperature and/or a time-temperature integral. Monophasic action potentials (MAPs) may be used by the preprocessor 40 to compute an average MAP, a change in MAP, and a percent change in MAP. Such MAPs may be obtained using, for example, a catheter with a diamond temperature split tip geometry, and may not be easily obtained from other tip geometries. The preprocessor 40 may also compute one or more Fourier transforms, or other transform(s) such as a wavelet transform, of the signals received by or preprocessed by the preprocessor 40. A Fourier transform transforms a time domain signal to a frequency domain signal so that the frequency domain signal can be analyzed as to frequency content. This enables, for example, characterization of changes in frequency characteristics of the MAP and/or electrograms.

Other computations by the preprocessor may include determining changes in electrocardiogram magnitude. For example, in some embodiments, the preprocessor 40 may perform a frequency analysis of an electrocardiogram signal, determine peak to peak amplitude of the electrocardiogram and/or absolute or percent change in voltage of the electrocardiogram. For monophasic action potentials, action potential duration (APD) at various times may be determined.

According to aspects of this disclosure, processing circuitry 70 may output a lesion property by outputting a relative indication of lesion formation. For example, processing circuitry 70 may be configured to output an indication of lesion depth as the lesion property. In this example, processing circuitry 70 may output, to be displayed on display 76, an absolute indication of lesion depth (e.g., according to a given measurement, such as millimeters). Additionally or alternatively, in the example of lesion depth, processing circuitry 70 may output, to be displayed on display 76, a relative indication of lesion formation (e.g., a percentage to a predetermined lesion depth, a color-coded indication of lesion depth, or the like). In such examples, the output may be used as a reference to determine when lesion formation is complete. For example, display 76 may display a green indication when ablation is beginning and the lesion depth is small, a yellow indication based on the lesion depth approaching a predetermined depth endpoint, and a red indication based on the lesion depth reaching the predetermine depth endpoint.

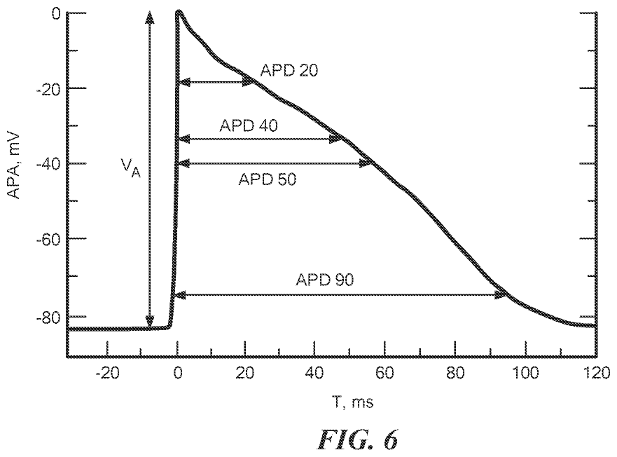

FIG. 6. shows one example of APDs of 30, 60 and 90% of repolarization, depolarization rate (time from baseline to peak amplitude) and amplitude that may be determined by the preprocessor 40. Other measurements input to the preprocessor 40 or quantities processed by the preprocessor 40 may include phase, rate of change in phase, amplitude, and rate of change in amplitude of, for example, impedance or other signal or measurement.

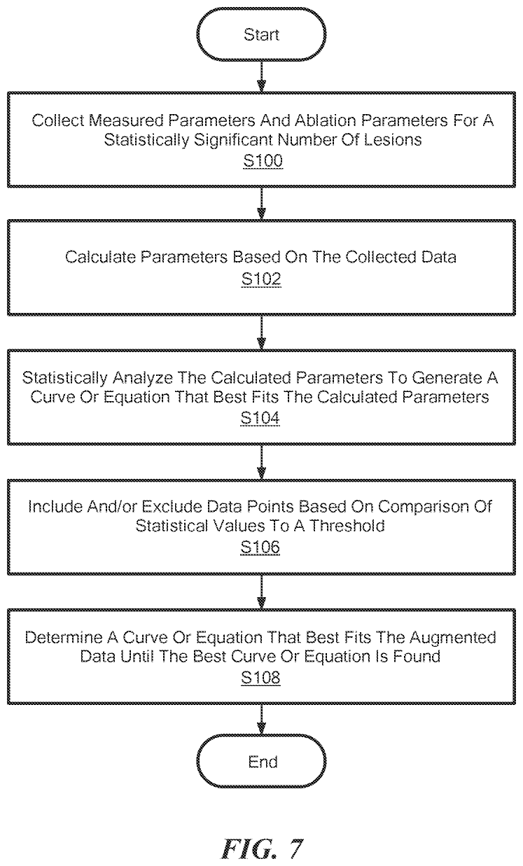

One or more of these calculated parameters are input to the lesion characteristics prediction (LCP) unit 42, which determines lesion characteristics based thereon. The LCP unit 42 performs an algorithm for calculating the lesion characteristics based on a previously performed statistical analysis of previously measured lesions. The statistical analysis may be performed using commercially available software such as Design Expert by StatEase®. Such software may be configured to receives measured data for a plurality of lesions and analyze the data to detect statistically significant relationships between the measured data and the characteristics of the lesions. The process of using statistical analysis software such as Design Expert to determine a model for determining lesion characteristics is described with reference to the flow chart of FIG. 7.

FIG. 7 is a flowchart of one example process of deriving an algorithm for lesion characterization. The process of FIG. 7 starts at Block S100 where measured parameters and ablation parameters are collected for a statistically significant number of lesions. The collected parameters are processed to produce the calculated parameters described above with respect to FIG. 5 (Block S102). The calculated parameters are then input to a statistical analysis algorithm which generates a curve and/or equation that best fits the calculated parameters (Block S104). Such an equation could be a polynomial, for example. Inputs to the statistical analysis are then included and/or excluded from the analysis successively based on statistical values such as p-values generated by the statistical analysis algorithm that are greater than or less than a threshold (Block S106). The statistical analysis algorithm outputs values called residuals, which are evaluated to determine whether another curve or equation provides a better fit to the remaining inputs. This is done for each lesion characteristic (e.g., surface area, volume, depth, transmurality). The example process of FIG. 7 also includes determining which calculated parameters are most significant for determining a particular lesion characteristic, and using the determination to find the best-fitting curve or equation to characterize the particular lesion characteristic (Block S108). How significant a calculated parameter is to determining a particular lesion characteristic may be determined by correlation. Thus, each input (such as, EGM, MAP, temperature, impedance magnitude and phase) may be correlated with lesion/output characteristics (depth, volume, surface area and transmurality). The results of the correlations may be evaluated in combinations to determine an optimal correlation equation for evaluating a specific lesion characteristic. Different optimal correlation equations may pertain to different lesion characteristics.

Once a best fitting curve or equation is found, it can be used to determine (or “predict”) lesion characteristics for a newly formed lesion, based on the pre-ablation, intra-ablation and post-ablation measurements.

For example, the statistical analysis may show that the most significant calculated parameters for characterizing lesion depth are change (or percent change) in impedance magnitude, change (or percent change) in peak to peak impedance magnitude, and an average MAP. As another example, the statistical analysis may show that the most significant calculated parameters for characterizing lesion transmurality are change (or percent change) in impedance magnitude and the change (or percent change) in impedance phase angle. As another example, the statistical analysis may show that the most significant calculated parameters for characterizing lesion surface area are change (or percent change) in impedance magnitude, change (or percent change) in peak to peak impedance magnitude, and change (or percent change) in impedance phase angle. Maximum temperature and a time-temperature integral may also be significant in determining lesion surface area. As yet another example, the statistical analysis may show that the most significant calculated parameters for characterizing lesion volume are change (or percent change) in impedance magnitude, change (or percent change) in peak to peak impedance magnitude, and change (or percent change) in impedance phase angle.

In some embodiments, at least one lesion property may be determined further based on at least one of a tissue thickness, a distance of a probe to the tissue of the lesion. In some embodiments, the at least one of the tissue thickness and the distance may be determined by at least one of an electro-anatomical mapping system, medical imaging and a dielectric method. Transmurality may then be determined a function of tissue thickness and lesion size.

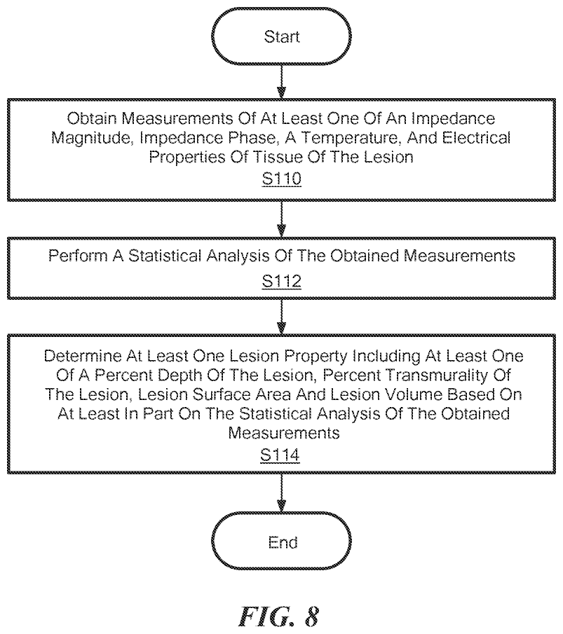

FIG. 8 is a flowchart of one example process of characterizing a lesion based on a plurality of measurements. The process may be performed by the data acquisition system 32, including the processing circuitry 34 and/or the processor 38 including preprocessor 40 and LCP 42. The process includes obtaining, by the preprocessor 40, measurements of at least one of an impedance magnitude, impedance phase, a temperature, and electrical properties of tissue of the lesion (Block S110). The process also includes performing a statistical analysis of the obtained measurements (Block S112). The process also includes determining, via the LCP 48, at least one lesion property including at least one of a depth of the lesion, percent transmurality of the lesion, lesion surface area and lesion volume based on at least in part on the statistical analysis of the obtained measurements (Block S114).

In some embodiments, the electrical properties of the tissue of the lesion include at least one of electrocardiogram (EGM) waveform and monophasic action potential (MAP) waveform properties. In some embodiments, determining at least one lesion property includes calculating the depth of the lesion from an equation that includes as inputs at least one of: an impedance phase change over a range of impedance phase measurements; a peak to peak impedance magnitude change over a range of impedance magnitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion surface area from an equation that includes as inputs at least one of: a percent change in phase of a measured impedance; an average peak to peak value of post-ablation measured impedance; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating lesion volume from an equation that includes as inputs at least one of: an average of post ablation impedance magnitude measurements; an average of post ablation impedance phase measurements; an average of changes in peak to peak impedance amplitude measurements; changes in electrical properties of tissue of the lesion; and a temperature measurement. In some embodiments, the equation is determined based at least in part on a statistical analysis of a plurality of measurements of the at least one of an impedance magnitude, impedance phase, a temperature, and changes in electrical properties of tissue of the lesion. In some embodiments, determining at least one lesion property includes calculating a transmurality of the lesion from an equation that includes as inputs at least one of: a percent change in impedance magnitude; a percent change in impedance phase; a maximum temperature measurement; changes in electrical properties of tissue of the lesion; and a sum of temperature measurements. In some embodiments, the equation is determined based at least in part on a statistical analysis of a plurality of the measurements of the at least one of an impedance magnitude, impedance phase, a temperature, and changes in electrical properties of tissue of the lesion. In some embodiments, determining at least one lesion property includes applying one of a Fourier transform, a filter and a wavelet matching algorithm to at least one of the obtained measurements. In some embodiments, determining at least one lesion property is further based on at least one of a tissue thickness, a distance of a probe to the tissue of the lesion. In some embodiments, at least one of the tissue thickness and the distance is determined by at least one of an electro-anatomical mapping system, medical imaging and a dielectric method.

It should be understood that various aspects disclosed herein may be combined in different combinations than the combinations specifically presented in the description and accompanying drawings. It should also be understood that, depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, it should be understood that the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope of the following claims.

What is claimed is:

1. A method comprising:
obtaining measurements of a lesion, the measurements comprising a temperature and at least one of an impedance magnitude, impedance phase, and electrical properties of tissue of the lesion;
performing a statistical analysis of the obtained measurements, wherein the statistical analysis does not include a force measurement;
determining at least one lesion property including at least one of a depth of the lesion, percent transmurality of the lesion, lesion surface area and lesion volume based at least in part on the statistical analysis of the obtained measurements; and
outputting the at least one lesion property.

2. The method of claim 1, wherein the electrical properties of the tissue of the lesion include at least one of electrocardiogram (EGM) waveform and monophasic action potential (MAP) waveform properties.

3. The method of claim 1, wherein determining at least one lesion property includes calculating the depth of the lesion from an equation that includes as inputs at least one of:
an impedance phase change over a range of impedance phase measurements;
a peak to peak impedance magnitude change over a range of impedance magnitude measurements;
changes in electrical properties of tissue of the lesion; and
a temperature measurement.

4. The method of claim 3, wherein the equation is determined based at least in part on a statistical analysis of a plurality of measurements of the at least one of an impedance magnitude, impedance phase, a temperature, and changes in electrical properties of tissue of the lesion.

5. The method of claim 1, wherein determining at least one lesion property includes calculating lesion surface area from an equation that includes as inputs at least one of:
a percent change in phase of a measured impedance;
an average peak to peak value of post-ablation measured impedance;
changes in electrical properties of tissue of the lesion; and
a temperature measurement.

6. The method of claim 5, wherein the equation is determined based at least in part on a statistical analysis of a plurality of measurements of the at least one of an impedance magnitude, impedance phase, a temperature, and changes in electrical properties of tissue of the lesion.

7. The method of claim 1, wherein determining at least one lesion property includes calculating lesion volume from an equation that includes as inputs at least one of:
an average of post ablation impedance magnitude measurements;
an average of post ablation impedance phase measurements;
an average of changes in peak to peak impedance amplitude measurements;
changes in electrical properties of tissue of the lesion; and
a temperature measurement.

8. The method of claim 7, wherein the equation is determined based at least in part on a statistical analysis of a plurality of measurements of the at least one of an impedance magnitude, impedance phase, a temperature, and changes in electrical properties of tissue of the lesion.

9. The method of claim 1, wherein determining at least one lesion property includes calculating a transmurality of the lesion from an equation that includes as inputs at least one of:
a percent change in impedance magnitude;
a percent change in impedance phase;
a maximum temperature measurement;
changes in electrical properties of tissue of the lesion; and
a sum of temperature measurements.

10. The method of claim 9, wherein the equation is determined based at least in part on a statistical analysis of a plurality of the measurements of the at least one of an impedance magnitude, impedance phase, a temperature, and changes in electrical properties of tissue of the lesion.

11. The method of claim 1, wherein determining at least one lesion property includes applying one of a Fourier transform, a filter and a wavelet matching algorithm to at least one of the obtained measurements.

12. The method of claim 1, wherein determining at least one lesion property is further based on at least one of a tissue thickness, a distance of a probe to the tissue of the lesion,

13. The method of claim 12, wherein at least one of the tissue thickness and the distance is determined by at least one of an electro-anatomical mapping system, medical imaging and a dielectric method.

14. The method of claim 1, wherein outputting the at least one lesion property comprises outputting a relative indication of lesion formation.

15. The method of claim 1, wherein the obtaining the measurements, the performing the statistical analysis, the determining the at least one lesion property and the outputting the at least one lesion property occur a plurality of times during an ablation procedure.

16. A lesion characterization system for characterizing a lesion based on a plurality of measurements, the system comprising:
processing circuitry configured to:
obtain measurements of at least one of an impedance magnitude, impedance phase, a temperature, and electrical properties of tissue of the lesion; and
perform a statistical analysis of the obtained measurements, wherein the statistical analysis does not include a force measurement;
determine at least one lesion property including at least one of a depth of the lesion, percent transmurality of the lesion, lesion surface area, lesion volume, and lesion contiguity based at least in part on the statistical analysis of the obtained measurements; and
output the at least one lesion property.

17. The system of claim 16, wherein the electrical properties of the tissue of the lesion include at least one of electrocardiogram (EGM) waveform and monophasic action potential (MAP) waveform properties.

18. The system of claim 16, wherein determining at least one lesion property based on the at least one of the obtained measurements includes calculating the depth of the lesion from an equation that includes as inputs at least one of:
an impedance phase change over a range of impedance phase measurements;
a peak to peak impedance magnitude change over a range of impedance magnitude measurements;
changes in electrical properties of tissue of the lesion; and
a temperature measurement.

19. The system of claim 18, wherein the equation is determined based at least in part on a statistical analysis of a plurality of measurements of the at least one of an impedance magnitude, impedance phase, a temperature, and electrical properties of tissue of the lesion.

20. The system of claim 16, wherein determining based on at least one of the obtained measurements includes calculating lesion surface area from an equation that includes as inputs at least one of:
a percent change in phase of a measured impedance;
an average peak to peak value of post-ablation measured impedance;
changes in electrical properties of tissue of the lesion; and
a temperature measurement.

21. The system of claim 20, wherein the equation is determined based at least in part on a statistical analysis of a plurality of measurements of the at least one of an impedance magnitude, impedance phase, a temperature, and changes in electrical properties of tissue of the lesion.

22. The system of claim 16, wherein determining based on at least one of the obtained measurements includes calculating lesion volume from an equation that includes as inputs at least one of:
an average of post ablation impedance magnitude measurements;
an average of post ablation impedance phase measurements;
an average of changes in peak to peak impedance amplitude measurements;
changes in electrical properties of tissue of the lesion; and
a temperature measurement.

23. The system of claim 16, wherein to output the at least one lesion property, the processing circuitry is configured to output a relative indication of lesion formation.

24. The method of claim 16, wherein the processing circuitry is configured to obtain the measurements, perform the statistical analysis, determine the at least one lesion property and output the at least one lesion property a plurality of times during an ablation procedure.
